(19) United States
(12) Patent Application Publication
(10) Pub. No.: US 2003/0229306 A1
(43) Pub. Date: Dec. 11, 2003

(54) Nasal lavage system and method

(76) Inventor: Roger Sherman, Crested Butte, CO (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/401,918

(22) Filed: Mar. 28, 2003

Related U.S. Application Data
(60) Provisional application No. 60/368,875, filed on Mar. 29, 2002.

(51) Int. Cl. A61M 31/00; A61M 37/00

(52) U.S. Cl. 604/93.01

(57) ABSTRACT

A nasal lavage device having a reservoir configured to hold a quantity of lavage solution. A spout is coupled to the reservoir operates to conduct the lavage solution from the reservoir to a spout end. The lavage system comprises alone or in combination an interchangeable tip assembly and a check valve mechanism. The tip assembly detachably couples to the spout end and is configured to fit in contact with a user's nose so as to enable lavage solution to be conducted from the reservoir, through the spout, through the tip to the user's nose. The check valve is located between the reservoir and the tip and operable to inhibit fluid backflow from the tip to the reservoir.

### RELATED APPLICATIONS

This application claims the benefit of U.S. Provisional Application No. 60/368,875, filed Mar. 29, 2002, which is incorporated herein by reference.

### BACKGROUND OF THE INVENTION

1. Field of the Invention

The present invention relates, in general, to personal hygiene, and, more particularly, to systems, apparatus and methods for performing nasal lavage to cleanse the nasal cavity.

2. Relevant Background

The human body is presented with an increasingly complex quantity and variety of contaminants. The prevalence of macro-scale contaminants such as dust, soot, and ash is being addressed by environmental regulation, but continue to be a significant health issue. Micro-scale and chemical contaminants are more difficult to detect and regulate and so human exposure continues to increase. Moreover, biological contaminants such as bacteria and viruses, including biotoxins used for military and terrorist purposes, present an increasing health risk worldwide.

The human nasopharyngeal system has evolved to protect the human organism by trapping contaminants such as viruses, bacteria, pollutants and the like. These contaminants are trapped by a system of mucous-lined passageways. Nasal and nasopharyngeal cavities are common places for holding environmental allergens, such as pollen, fungal spores, animal body-originated dustings and volatile chemicals. These harmful agents cause allergic reactions and other health consequences.

The nasopharyngeal system is not, however, completely self-cleaning. As a result, many disease-causing agents exist in the nasal and nasopharyngeal cavities. Exposure to these pathogens may be acute or chronic, but in either case the quantity or proportion of pathogens trapped increases unless the nasopharyngeal system is cleansed. As the loading of these pathogenic agents in the mucous increases to clinically significant levels, health and comfort are negatively impacted. Hence, a need exists for efficient tools and methods for cleaning the nasopharyngeal system as a part of routine personal hygiene.

[refer to Breathing,]

With respect to an individual's health, even though the nasopharyngeal system may trap contaminants, if they are not removed they can readily lead to health problems. First, local tissues in the sinuses may be impacted resulting in irritation, infection, or more serious consequences. Second, contaminants may progress farther into the human system (i.e., vertical transmission), effectively using the nasopharyngeal system as a launching platform. For example, viruses trapped in the sinuses readily migrate from the upper respiratory tract to the lower respiratory tract of the carrier.

Similarly, trapped viruses also enable spreading of the pathogens to other people through sneezing and/or coughing, for example (i.e., horizontal transmission). Hence, it is beneficial to have a practical system and/or method to remove these disease causing agents from the human body. In a manner similar to brushing one's teeth, nasal and nasopharyngeal cleaning in the general population will have a number of health benefits.

Several methods have been reported to be useful in cleaning nasal and nasopharyngeal cavities. These generally involve an isotonic saline solution under pressure flowing into one nostril, passing through the sinuses and being discharged from the second nostril. These pressurized systems increase the risk that contaminants will be forced into adjacent areas such as the Eustachian tubes, in effect defeating the purpose of the natural protection mechanisms that exist.

A technique called “neti”, was developed ages ago in India. The device, which has changed little since its conception, is a tea-pot like structure that enables a person to gently run a rinse solution through the nasal passages without pressure. Although this simple device performs well, the conventional design allows reverse flow from the user's nasal cavity into the reservoir, potentially contaminating the rinse solution. Moreover, the device itself may become contaminated. Further, after each use the device must be carefully cleaned as the device comes into intimate contact with the user's nostrils and exterior nasal passages. Accordingly, a need exists for a system and method of providing nasal lavage that obtains the benefits of simplicity and un-pressurized operation, while at the same time overcoming the limitations associated with multiple users and contamination of the device and/or lavage solution.

### SUMMARY OF THE INVENTION

Briefly stated, the present invention involves a nasal lavage device having a reservoir configured to hold a quantity of lavage solution. A spout is coupled to the reservoir operates to conduct the lavage solution from the reservoir to an end of the spout. The lavage system comprises alone or in combination an interchangeable tip assembly and a check valve mechanism. The tip assembly detachably includes a spout-end 203 that couples to the spout 103 and is configured to fit in contact with a user's nose so as to enable lavage solution to be conducted from the reservoir, through the spout, through the tip to the user's nose. The check valve is located between the reservoir and the tip and operable to inhibit fluid backflow from the tip to the reservoir.

In another aspect, the present invention involves an interchangeable tip for a nasal lavage system. An exterior sealing surface creates a seal to a user's nostril. A channel formed in the tip conducts fluid into the user's nostril. An attachment mechanism located at one end of the interchangeable tip operates to removably attach the tip to a reservoir of lavage fluid. Each interchangeable tip can be associated with a particular user thereby enabling the remaining portions of the lavage system to be hygienically shared amongst several people. The nasal lavage tip may also include a check valve integrated within the tip to prevent backflow of fluid from the nostril into the reservoir through the channel.

In still another aspect, the present invention involves a disposable nasal lavage system using a disposable reservoir operable to hold a quantity of lavage solution. A tip is coupled to the reservoir having a channel formed therein and operable to conduct fluid into the user's nostril from the reservoir. Preferably, a venting mechanism in the reservoir releases vacuum created by dispensing the lavage solution from the tip.

### BRIEF DESCRIPTION OF THE DRAWINGS

FIG. 1 shows an exemplary nasal lavage system in which the present invention is implemented;

FIG. 2 shows the system of FIG. 1 in cross-section;

FIG. 3A through FIG. 3D show various alternative embodiments of interchangeable tip mechanisms in accordance with the present invention;

FIG. 4 illustrates a portion of an exemplary valve used in the embodiment of FIG. 3A;

FIG. 5 illustrates a first disposable implementation of the present invention;

FIG. 6 illustrates a second disposable implementation of the present invention; and

FIG. 7 illustrates a second disposable implementation of the present invention.

### DETAILED DESCRIPTION OF THE PREFERRED EMBODIMENTS

In present invention involves a system for providing nasal lavage in which a liquid is introduced into the nasal cavity under a low pressure or gravity feed system to irrigate the nasal cavities and surrounding areas. The dispenser in accordance with the present invention is primarily intended for use by the general public in a program of periodic personal hygiene, but could also be used by health care providers in doctor's offices, dentist offices, hospitals, and the like.

The present invention is illustrated and described in terms of a lavage system having an interchangeable tip or nose piece such that the portion of the device that comes into contact with any individual is, like a toothbrush, intended for use by a single person. The interchangeable tip may be readily removed, cleaned and stored. Preferably, a valve component is used in combination with the interchangeable nose piece to further inhibit backflow of liquid into the shared components of the system. These features inhibit contamination of shared components of the lavage system.

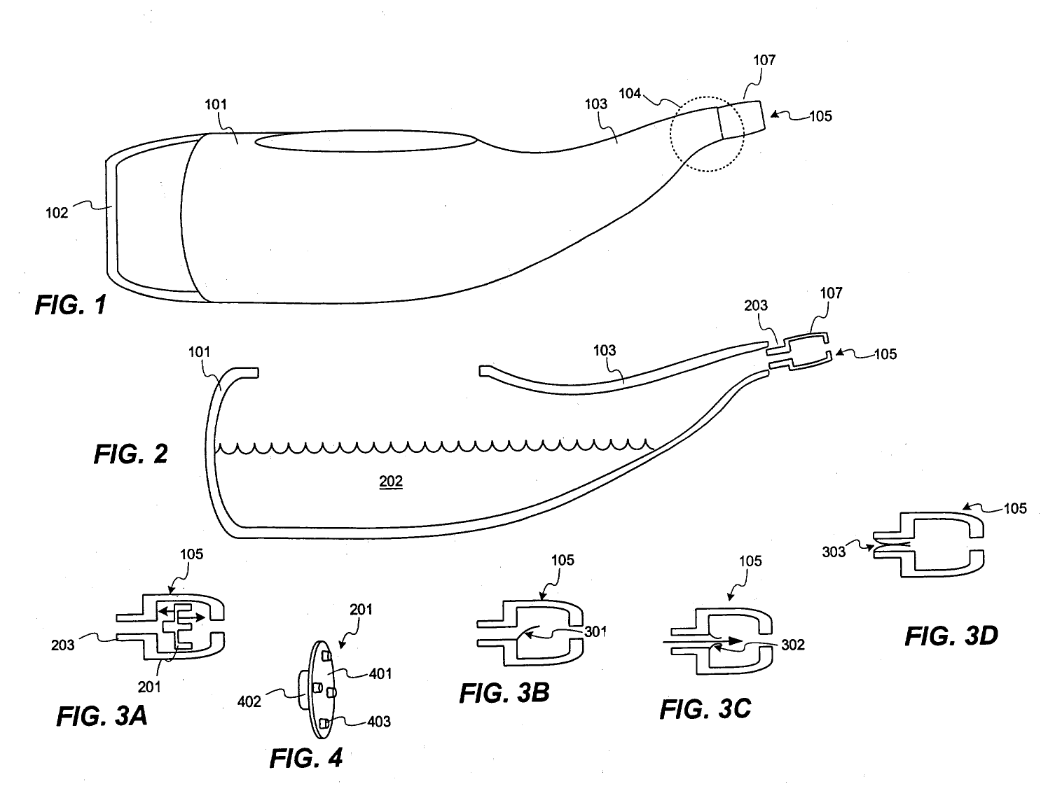

FIG. 1 and FIG. 2 show an exemplary nasal lavage system in which the present invention is implemented. A reservoir 101 is designed to hold a quantity of lavage solution 202 (shown in FIG. 2) and preferably includes a handle 101 to improve handling and ease of use. A spout 103 provides a channel that conducts lavage solution 202 to a tip end of the spout when reservoir 101 is tilted. Significantly, reservoir 101 is not pressurized and the flow of lavage solution 202 is by gravity feed under the control of a user. A tip 105 having nasal contact surfaces 107 is attached to the tip-end of spout 103. In use, a person fills reservoir 101 with solution 202, then places tip 105 in contact with a nostril to make a tight or loose seal with the nostril. Lavage fluid 202 is then conducted into the nostril to wash the nasal cavities and surrounding areas.

One advantage of the various features of the present invention is that much of the nasal lavage system can be hygienically shared by multiple users. This enables a family, for example, to have a single nasal lavage system rather than each person having their own separate system. Specifically, tip 105 is made to be detachable so that each user may have their own tip 105. Alternatively, tip 105 may be discarded after each use. In some embodiments, an anti backflow valve or check valve is implemented in region 104. The valve may be implemented in the tip-end of spout 103, but is more preferably implemented in a tip 105 itself. This feature of the present invention prevents backflow of fluid from tip 105 to spout 101 and/or reservoir 101.

Reservoir 101 and spout 103 are formed as an integral structure in the exemplary implementation, although this is not required. Alternatively, spout 103 may be made detachable instead of or in addition to tip 105 so that the lavage system can be broken down for thorough cleaning in a dishwasher, for example. Reservoir 101 and spout 103 may be made of metal such as stainless steel, copper, silver, alloys and the like, plastics, ceramics or other water impermeable and hygienic materials.

Reservoir and spout 103 may take on a wide variety of shapes such as the horizontally elongated shape shown in FIG. 1 and FIG. 2, or more upright shapes having a spout that resembles a plant watering cans or pots, or any generally spouted-teapot shaped vessel. The construction may be rigid, semi-rigid, or flexible. In particular, it is contemplated that spout 103 may be formed of a formable tube-like material such as a wire-reinforced plastic or rubber. This allows spout 103 to be shaped and re-shaped to the desires of a user.

Lavage solution 202 typically comprises warm isotonic saline solution. However, many variations are possible. It has been found that buffering the solution with bi-carbonate may increase user comfort. Some believe that slightly acidic solutions comprising citric or tanic acid in very low concentration may improve germ killing capability. It is also contemplated that medicants such as steroids, anti-inflamatories, decongestants, and the like may be included alone or in combination in solution 202 to meet the needs of particular users.

In practice, lavage solution 202 may be supplied in a pre-mixed form that is simply poured into reservoir 101. Alternatively, a measured quantity of salt and/or medicine and/or buffering agents is mixed with water in reservoir 101 to form the solution each time the lavage system is used. In this case, it may be advantageous to provide a lid or other type of seal for the openings in the top of reservoir 101 and/or the spout 103 so that lavage solution 202 can be easily mixed by agitating reservoir 101 without spilling.

FIG. 3A through FIG. 3D show various alternative embodiments of interchangeable tip mechanisms in accordance with the present invention. In FIG. 3A a valve member 202 comprises a shaped plastic, rubber, metal, or other rigid or semi-rigid material. The valve member 201 is shown in greater detail in FIG. 4. When tipped forward, fluid can spill around the base 401 which is restrained from making a sealing contact with the body of tip 105 by several projections 403. When tipped backward, or under the influence of reverse flow, sealing surface 402 of valve member 201 seals back against a spout-end of tip 105 sealing surface 402 may be flat or tapered to make an adequate seal, or may have an outside diameter that is less than the inside diameter of spout-end 203 of tip 105.

FIG. 3B illustrates an alternative valve arrangement using a hinged flap 301 to provide unidirectional flow. Flap 301 may comprise a membrane of the same material used to form tip 105, or may comprise a sheet or membrane of almost any material that can be attached to tip 105 in a manner that enables hinged action. FIG. 3C illustrates a modification in which valve 302 is formed by a membrane having a slit or similar self-closing opening formed therein. In yet another alternative, FIG. 3D illustrates a valve mechanism 303 that can be mounted on the spout-end of tip 105. For example, valve mechanism 303 could be readily attached to or inserted in a formed tip 105 and may form a part of the mechanism that attaches tip 105 to spout 103. A wide variety of valve types and configurations are known, and many variations and equivalents to the specific teachings herein will be appropriate for particular applications.

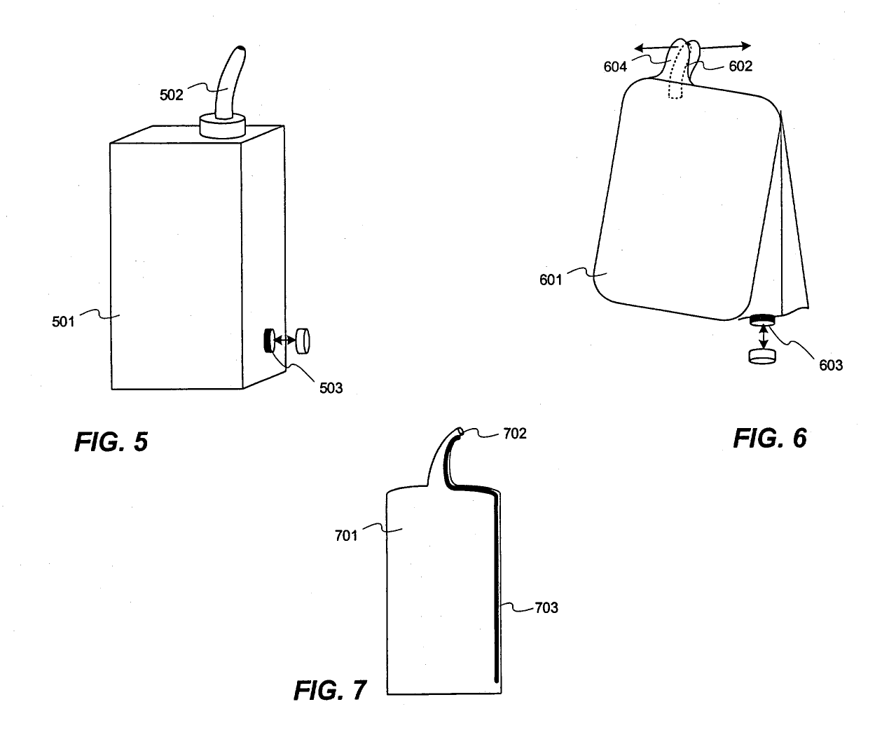

A disposable implementation is expected to be particularly valuable for use in office first aid kits, vending machine applications, and by travelers. FIG. 5 illustrates a first disposable implementation of the present invention. A disposable reservoir 501 may be formed of a plastic or paperboard box as is often used for disposable drink containers. Such containers are easily sealed to protect the sterility of the lavage solution contained therein. A shaped tip 502 is attached at one end of reservoir 501 and may or may not include a valve mechanism as described hereinbefore. Preferably, a vent such as screw-top sealable vent 503 is used to relieve vacuum build up during use. Alternatively, vent 503 may be implemented with a simple pull-tab type device that allows the user to regulate the flow of liquid by selectively blocking vent 503 with a thumb or finger.

FIG. 6. illustrates a second disposable implementation of the present invention using a collapsible bag dispenser. Reservoir 601 is implemented with a collapsible paper or mylar bag and preferably comprises a material that is deformable, but not elastic. In this manner, lavage solution within reservoir 601 is under little or no pressure during use. A tip 602 is coupled to reservoir 601 and operates in a manner similar to that described above. A sealable flap 604 is preferably included that envelops tip 602 during shipping and handling. A user may release tip 602 by pulling the sealed tabs 604 apart to expose the sterile tip 602. A vent, such as vent 503 shown in FIG. 5, may also be included in the design shown in FIG. 6.

FIG. 7 illustrates a third disposable implementation of the present invention using a collapsible bag dispenser. Reservoir 701 is implemented with a collapsible paper bag, plastic bag, mylar bag, or the like and preferably comprises a material that is deformable, but not elastic. In this manner, lavage solution within reservoir 701 is under little or no pressure during use as in the case of the embodiment shown in FIG. 6. A tip 702 is coupled to reservoir 701 and operates in a manner similar to that described above. A vent tube 703 has one end that couples to an area on or near tip 702 and leads inside reservoir 701. Another end of vent tube 703 is located near a bottom of reservoir 701. In operation, when reservoir 701 is tipped sideways or upside down to dispense the lavage solution contained in reservoir 701, vent tube 703 provides a conduit for pressure equalization within reservoir 701. Preferably, a cap or sealing device (not shown) provides a sanitary seal for both the exit portion of tip 702 and vent tube 703 so that the device shown in FIG. 7 is readily shipped and handled while maintaining the contents of reservoir 701 in a sanitary condition. Vent tube 703 may exit reservoir 701 at any convenient location and may be provided as, for example, a straw that is inserted through a wall of reservoir 703 just prior to use.

In an alternative mode of operation, disposable reservoirs 501 and 601 may be shipped with a pre-measured quantity of dry salt, medicines, or other materials used in lavage solution 202. A user will then add warm water and mix the solution before use. In one example, vent 501 can be used as a means for adding water to reservoir 601, then sealed. The sealed reservoir 501/601 can then be agitated to mix the solution before use. It is contemplated that a variety of lavage solution mixtures may be provided, each in a different labeled container so as to be readily identified by a user. By supplying the lavage solution 202 in a pre-measured, dehydrated form, the package is convenient to transport and use.

Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as hereinafter claimed.

We claim:

1. A nasal lavage device comprising:
a reservoir configured to hold a quantity of lavage solution;
a spout coupled to the reservoir and operable to conduct the lavage solution from the reservoir to a spout end; and
a tip assembly detachably coupled to the spout end and configured to fit in contact with a user's nose so as to enable lavage solution to be conducted from the reservoir, through the spout, through the tip to the user's nose.

2. The nasal lavage device of claim 1 further comprising a check valve located between the reservoir and the tip and operable to inhibit fluid flow from the tip to the reservoir.

3. The nasal lavage device of claim 2 wherein the valve is integrated with the detachable tip assembly.

4. The nasal lavage device of claim 2 wherein the valve is integrated with the spout.

5. The nasal lavage device of claim 1 wherein the spout comprises a flexible fluid conduit that can be shaped by a user.

6. The nasal lavage device of claim 1 wherein the solution comprises a saline solution.

7. A method for performing nasal lavage comprising:
filling a reservoir with a lavage solution;
attaching an interchangeable tip to the reservoir;
sealing the tip against a nose; and
conducting the lavage solution from the reservoir through the tip to the nose.

8. The method of claim 7 wherein filling the reservoir comprises filling the reservoir with a premixed isotonic saline solution.

9. The method of claim 7 wherein filling the reservoir comprise mixing a dry salt solution with warm water in the reservoir and agitating the mixture until the salt solution is dissolved.

10. The method of claim 7 further comprising removing and cleaning the interchangeable tip separate from the reservoir.

11. A nasal lavage device comprising:
a reservoir configured to hold a quantity of lavage solution;
a spout coupled to the reservoir and operable to conduct the lavage solution from the reservoir to a spout end;
a check valve located between the reservoir and the tip and operable to inhibit fluid flow from the tip to the reservoir; and
a tip at the spout end and configured to fit in contact with a user's nose so as to enable lavage solution to be conducted from the reservoir, through the spout, through the tip to the user's nose.

12. The nasal lavage device of claim 11 wherein the check valve is integrated with the tip.

13. The nasal lavage device of claim 11 wherein the check valve is integrated with the spout end.

13. The nasal lavage device of claim 11 wherein the check valve is located in the spout closer to the reservoir than to the tip.

14. The nasal lavage device of claim 11 wherein the spout is detachably coupled to the reservoir.

15. The nasal lavage device of claim 11 wherein the tip is detachably coupled to the spout.

16. An interchangeable nasal lavage tip comprising:
an exterior sealing surface for creating a seal to a user's nostril;
a channel formed in the tip and operable to conduct fluid into the user's nostril;
an attachment mechanism located at one end and operable to removable attach the tip to a reservoir of lavage fluid.

17. The nasal lavage tip of claim 16 further comprising a check valve integrated within the tip to prevent backflow of fluid from the nostril into the reservoir through the channel.

18. A disposable nasal lavage system comprising:
a disposable reservoir operable to hold a quantity of lavage solution;
a tip coupled to the reservoir having a channel formed therein and operable to conduct fluid into the user's nostril from the reservoir;
a venting mechanism in the reservoir and operable to release vacuum created by dispensing the lavage solution from the tip.

19. The disposable nasal lavage system of claim 18 further comprising a check valve located between the reservoir and the tip and operable to inhibit fluid flow from the tip to the reservoir.

20. The disposable nasal lavage system of claim 18 wherein the disposable reservoir is pre-filled with quantity of lavage solution.

21. The disposable nasal lavage system of claim 18 wherein the disposable reservoir is pre-filled with quantity of salt and the lavage solution is made at the time of use by adding water to the disposable reservoir.

22. The disposable nasal lavage system of claim 21 wherein the water is added through the venting mechanism.

23. The disposable nasal lavage system of claim 21 wherein the disposable reservoir comprises a collapsible container.
